(19) United States
(12) Patent Application Publication
(10) Pub. No.: US 2012/0088958 A1
(43) Pub. Date: Apr. 12, 2012

(54) NON-FIBROUS BARRIER FOR ORGAN RETENTION

(76) Inventors: Steven J. Kubisen, Ellicott City, MD (US); Anthony P. Deasey, Severna Park, MD (US)

(21) Appl. No.: 13/166,616

(22) Filed: Jun. 22, 2011

Related U.S. Application Data
(60) Provisional application No. 61/392,462, filed on Oct. 12, 2010.

(51) Int. Cl. A61F 2/00

(52) U.S. Cl. 600/37

(57) ABSTRACT

A method of packing organs of a subject, the method including accessing an interior cavity of the subject, repositioning the organs to provide a surgical space in the abdominal cavity, and positioning a non-fibrous body against the organs to provide a barrier between the organs and the surgical space.

### CROSS-REFERENCE TO RELATED APPLICATIONS

The contents of U.S. Provisional Patent Application No. 61/392,462 died on Oct. 12, 2010, U.S. Provisional Patent Application 61/125,219 filed on Apr. 23, 2008, and PCT/US2009/002495 filed on Apr. 22, 2009, are hereby incorporated herein by reference in their entirety.

### BACKGROUND

1. Field of the Invention

The present invention relates generally to organ barriers, and more particularly, to a non-fibrous barrier for organ retention.

2. Related Art

Abdominal and pelvic laparotomies require displacement and retention of bowels or other organs to create a space that allows the surgeon to perform a surgical procedure. This procedure is referred to herein as bowel packing. The current bowel packing procedure used in the operating room today involves manually displacement of the bowels using surgical cotton sponges made of loose cotton fibers. Such fibers may remain in the abdominal cavity even after removal of the sponges, and can promote peritoneal inflammation, a major cause of post-operative adhesion formation. Furthermore, the sponges tend to dry out over the course of the surgical procedure, becoming abrasive and adhere to the bowels themselves, further contributing to the formation of adhesions.

### SUMMARY

According to one aspect of the present invention, there is provided a method of packing organs of a subject. The method comprises: accessing an interior cavity of the subject; repositioning the organs to provide a surgical space in the cavity; and positioning a non-fibrous body against the bowels to provide a barrier between the organs and the surgical space.

According to another aspect of the present invention, there is provided a surgical procedure. The procedure comprises: accessing organs of a subject; positioning a non-fibrous body in contact with the organs; and absorbing body fluids proximate to the organs with the absorbant non-fibrous body.

### BRIEF DESCRIPTION OF THE DRAWINGS

Embodiments of the present invention are described below with reference to the attached drawings, in which:

FIG. 1 is a perspective view of a non-fibrous body, in accordance with embodiments of the present invention;

FIG. 2 is a perspective view of a non-fibrous body, in accordance with embodiments of the present invention;

FIG. 3 is a perspective view of a non-fibrous body, in accordance with embodiments of the present invention;

FIG. 4A is a perspective view of the bowels of an average adult human;

FIG. 4B depicts exemplary use of a non-fibrous body, in accordance with embodiments of the present invention;

FIG. 5A is a flowchart of an exemplary method, in accordance with embodiments of the present invention; and

FIG. 5B is a flowchart of an exemplary method, in accordance with embodiments of the present invention.

### DETAILED DESCRIPTION

Aspects of the present invention are generally directed to a method for packing bowels or other organs of a subject through the use of a non-fibrous body. More particularly, the subject's bowels are repositioned within the interior of the abdominal cavity to provide a surgical space, and one or more non-fibrous bodies are placed against the bowels. The non-fibrous bodies provide a barrier between the bowels and the surgical space, thereby maintaining the surgical space during abdominal surgery. Because the bodies are non-fibrous, little or no fibers remain on the bowels after removal of the bodies from the subject.

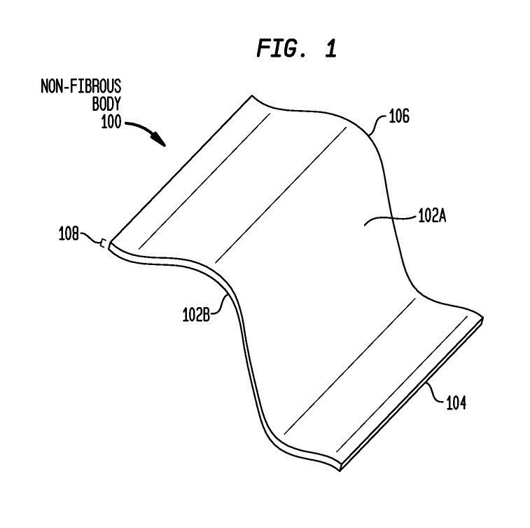

FIG. 1 is a perspective view of an exemplary embodiment of a non-fibrous body 100 in accordance with embodiments of the present invention. Similar to a cotton cloth, body 100 is substantially conformable so that a surgeon may manipulate the body for positioning abutting the subject's bowels. Additionally, body 100 may be formed from different materials. In certain embodiments, body 100 is a synthetic material, such as rayon, nylon, acrylic, olefin, or polyester. In such embodiments, body 100 may have material and absorbent properties that are similar to a synthetic chamois. In some embodiments, non-fibrous absorbent body 100 is combination of synthetic and natural chamois.

In further embodiments, body 100 is a natural material, such as chamois or silk. In such embodiments, body 100 may have material and absorbent properties that are similar to a natural chamois.

In the exemplary arrangement of FIG. 1, body 100 has two generally rectangular shaped surfaces 102 and a thickness 108. The surfaces have a length 106, and width 104. As such, body 100 essentially has the shape of a conventional towel.

As would be appreciated, the length 106, width 104 and thickness 108 of body 100 may vary depending on, for example, the subject (age, size, etc.), type of surgery, surgical approach (laproscopic or laparotomy), etc. In a specific laparotomy abdominal surgery, body 100 has a length 106 of approximately 6 to 10″, a width 104 of approximately 4 to 6″, and a thickness 108 of approximately ⅓ to ½″ In a specific laproscopic abdominal procedure, body 100 has a length 106 of approximately 8″, a width 104 of approximately 5″, and a thickness 108 of approximately ⅓″ It would be appreciated that these dimensions are provided for illustration purposes only, and other sizes are within the scope of the present invention.

As noted above, in the embodiments of FIG. 1, body 100 has a generally rectangular shape. In other embodiments, body may be, for example, oval, elliptical or star shaped. In some embodiments, body 100 may include notches or the like to provide clearance for portions of the subject's anatomy such as, for example, the spine. Such shapes and configurations are merely for illustration, and surgical procedures in accordance with embodiments of the present invention may use any shape of an non-fibrous body.

As noted above, when fibers adhere to the bowels and remain after surgery, the fibers contribute to the formation of post-operative adhesions. Because body 100 is a non-fibrous material, surgical procedures in accordance with embodiments of the present invention that use non-fibrous bodies have the advantage that no fibers are present that will adhere to the bowels during the surgery. In certain additional embodiments, body 100 is absorbent.

Furthermore, as noted above, conventional cotton sponges may be abrasive when moved against the bowles so as to further contribute to the formation of adhesions. As such, in embodiments of the present invention, fibrous body 100 does not include edges or surfaces that could potentially be abrasive to the bowels during surgery. In such an arrangement, the edges of body 100 are rounded to be substantially un-abrasive.

FIG. 1 illustrates embodiments of the present invention in which body 100 is similar to a towel. That is, the length 106 and width 104 of body 100 is substantially greater than the thickness 108. As previously noted, other shapes, sizes and configurations are within the scope of the present invention. It would also be appreciated that other types of non-fibrous bodies may be used in embodiments of the present invention. For example, in one alternative embodiment, a non-fibrous body in accordance with embodiments of the present invention is a foam material, such as a foam wound dressing. In one such embodiment, the foam is an absorbent, open-celled foam, such as a silicone foam or an elastomeric foam. In another such embodiment, the foam is a closed cell foam that is non-absorbent.

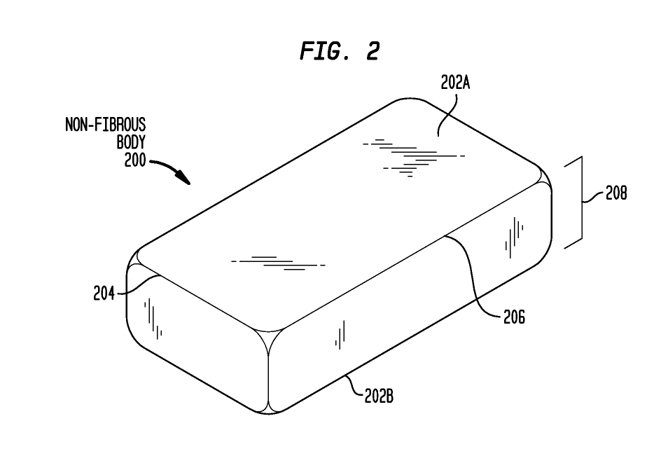

FIG. 2 is a perspective view of an alternative embodiment in which a non-fibrous body 200 has a block or sponge shape. More specifically, body 200 has a length 206 and width 204 that form a rectangular shape, but also as a significant thickness 208 that is substantially greater than thickness 108 of FIG. 1.

In a specific laparotomy abdominal surgery, body 200 has a length 206 of approximately 7″, a width 204 of approximately 5″, and a thickness 208 of approximately ½″ It would be appreciated that these dimensions are provided for illustration purposes only, and other sizes are within the scope of the present invention.

Similar to body 100, body 200 body 100 is substantially conformable so that a surgeon may manipulate the body for positioning abutting the subject's bowels. Additionally, body 200 may be formed from the same, or different material as used for body 100. In one specific arrangement, body 200 is formed from silicone foam.

In the exemplary arrangement of FIG. 2, body 200 has a generally rectangular shape. In other embodiments, body 200 may be, for example, oval, elliptical or star shaped. In some embodiments, body 200 may include notches or the like to provide clearance for portions of the subject's anatomy such as, for example, the spine. Such shapes and configurations are merely for illustration, and surgical procedures in accordance with embodiments of the present invention may use any shape of an non-fibrous absorbent body.

As noted above, when fibers adhere to the bowels and remain after surgery, the fibers contribute to the formation of post-operative adhesions. Because body 200 is a non-fibrous material, surgical procedures in accordance with embodiments of the present invention that use non-fibrous bodies have the advantage that no fibers are present that will adhere to the bowels during the surgery.

Furthermore, as noted above, conventional cotton sponges may be abrasive so as to further contribute to the formation of adhesions. As such, in embodiments of the present invention, fibrous body 200 does not include edges or surfaces that could potentially be abrasive to the bowels during surgery. In such an arrangement, the edges of body 200 are rounded to be substantially un-abrasive.

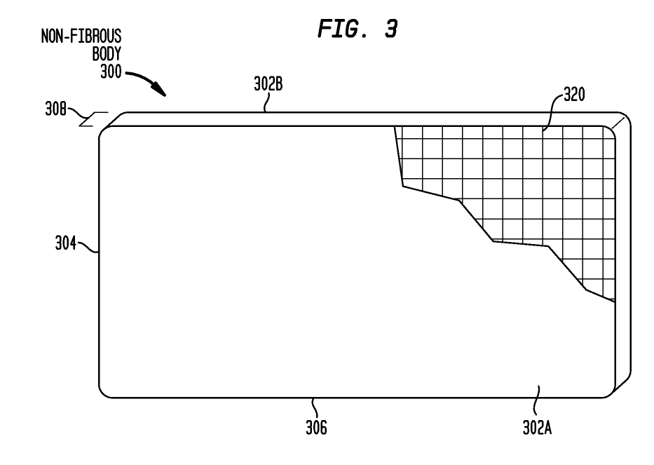

FIG. 3 is a perspective view of a non-fibrous body 300 in accordance with further embodiments of the present invention. Body 300 has two rectangular surfaces 302 each having a length 306 and a width 304. Body 300 also has a thickness 308.

In FIG. 3, a portion of surface 302A has been removed to show that body 300 further includes a reinforcement or support structure 320. In this embodiment, support structure 320 is a wire mesh that is embedded in a non-fibrous material. Wire mesh 320 may be embedded in the non-fibrous material by sewing or otherwise positioning one or more pieces of the non-fibrous material around the mesh. The non-fibrous material may be similar to the materials detailed above with reference to FIG. 1. In one specific arrangement, body 200 is formed from silicone foam or synthetic chamois.

FIG. 3 illustrates embodiments in which wire mesh 320 has a lattice pattern of symmetrical squares. It would be appreciated that other lattice patterns of different shapes are within the scope of the present invention. Different shapes and sizes of lattice patterns may provide non-fibrous body 320 with different levels of conformability, such as, for example, a degree of relative rigidity in one direction while providing a degree of relative flexibility in another direction.

Support structures in accordance with embodiments of the present invention may, in certain embodiments, be configured to add rigidity to assist in support of the bowels, but also be substantially conformable for proper positioning in the subject. This rigidity and conformability may be obtained through proper selection of the support material, shape or size of the supports, positioning of the supports within the non-fibrous material, etc. For example, support structures embedded in a non-fibrous material need not be a mesh arrangement as shown in FIG. 3. For example, a support structure may include independent members that extend across the full length and/or height of body 300, or only across a portion thereof. Such embodiments permit body 300, when functioning as a barrier, to have greater rigidity at certain areas, but have substantial conformability at other areas for positioning of the non-fibrous absorbent body. In other embodiments, the support structure may have portions that are more rigid than other portions and/or multiple structures of different rigidity may be used in the same non-fibrous absorbent assembly. It would be appreciated that a variety of sizes, shapes or numbers of support structures may be used in embodiments of the present invention. It is also noted that in lieu of or in addition to the reinforcement structure, the thickness of the non-fibrous body may be varied to achieve greater stiffness, as desired.

FIGS. 1-3 illustrative various embodiments of a non-fibrous body that may be used in methods of the present invention. In specific arrangements of the above embodiments, the non-fibrous absorbent bodies include a coating. The coating can be useful to prevent adhesion of the non-fibrous body to the bowel or to provide any other desirable surface property to the non-fibrous body. Alternatively, non-fibrous bodies may be coated with agents, for example, anti-microbial agents such as anti-viral agents or anti-bacterial agents. The use of such agents may be useful for the protection of the subject as well as the surgical staff and to reduce the possibility of transmission of infection from subjects infected with HIV, hepatitis, especially drug-resistant forms of hepatitis, methicillin resistant staphylococcus aureus (MERSA), etc.

It is noted that other methods of use of the non-fibrous bodies detailed herein are not limited to use with bowels, may also include methods for providing a barrier between other organs of a subject and a surgical space.

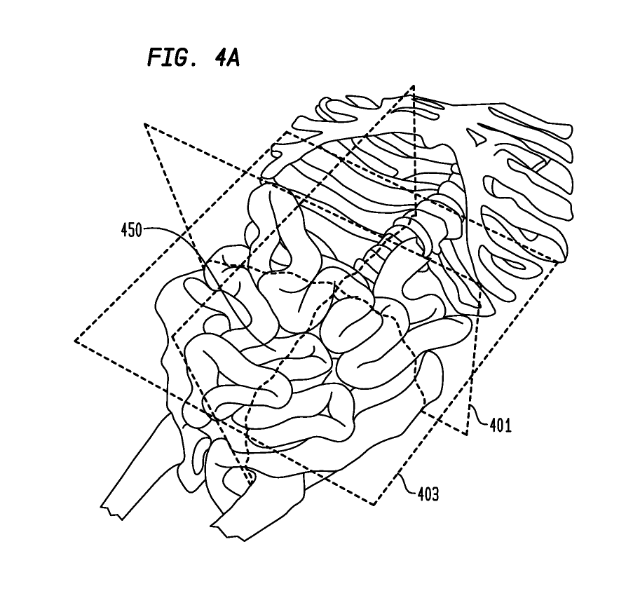

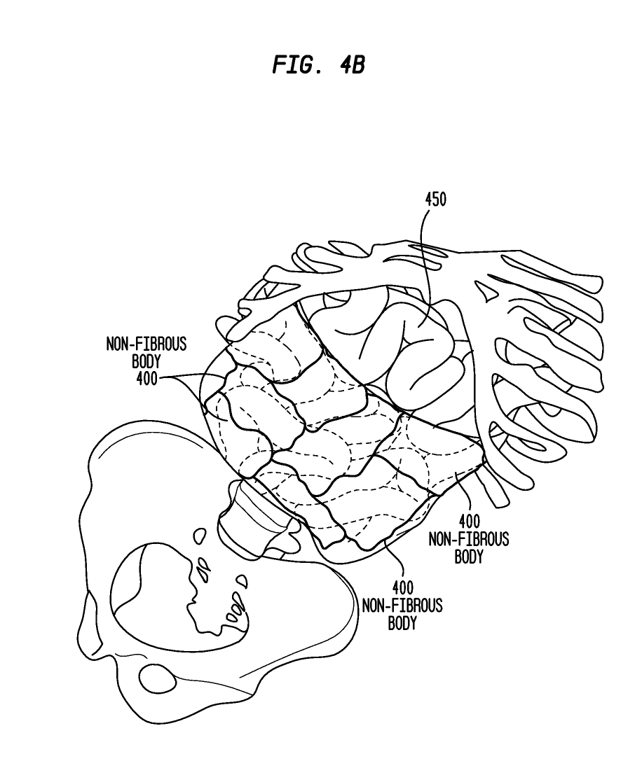

FIG. 4A is an illustration of an average human abdomen cavity containing bowels 450. FIG. 4A also illustrates a transverse plane 401 and coronal plane 403. Using measurements of the average human adult abdominal cavity, one may scale the size of the non-fibrous absorbent body(s) for use to provide a barrier between the bowels 400 and the surgical space. One may also scale the size of the non-fibrous absorbent body(s) for use to provide a barrier between the bowels of subjects other than an average adult human (human child, relatively large human adult, relatively small human adult, dog, cat, etc.). Data may be used to determine one or more sizes designed to fit at least 95% of the adult human population. Illustrative sizes were provided above.

FIG. 4B is an illustration of the cavity from FIG. 4A with a plurality of absorbent bodies 400 positioned in the subject. As shown, bodies 400 are positioned in the cavity so as to abut bowels 450. Bodies 400 may similar to any of the embodiments described above. Additionally, absorbent bodies 400 may all have the same or different configurations (shape, size, type, etc.). For example, some embodiments include a small size, a medium size and a large size non-fibrous absorbent body.

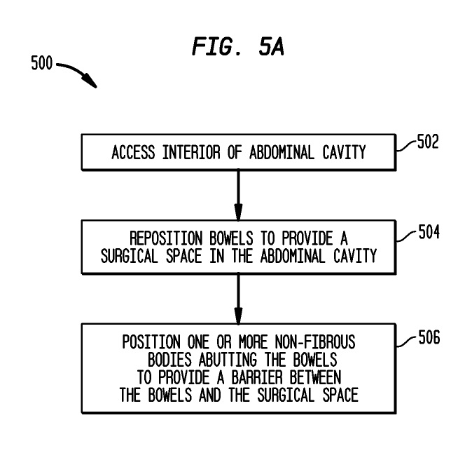

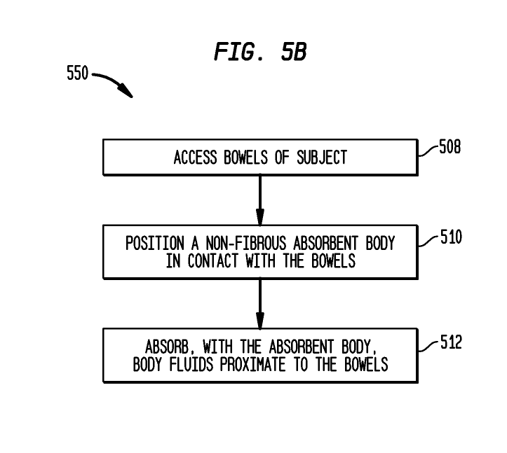

FIG. 5A is a flowchart of a method 500 for use of a non-fibrous body, in accordance with embodiments of the present invention. Method 500 begins at step 502 where access to the interior of a subject's abdominal cavity is obtained. That is, the surgeon or other medical professional gains access to the cavity through, for example, a laparoscopic procedure or a more invasive procedure, such as a laparotomy. At step 504, the surgeon repositions the bowels to provide a surgical space in the abdominal cavity of the subject. This may be done by hand, in the case of a laparotomy, or the case of a laparoscopic procedure, remotely using a probe or the like extending through a gel port or incision. At step 506, one or more non-fibrous bodies are positioned abutting the bowels of the subject in such a manner that the non-fibrous bodies provide a barrier between the bowels and the surgical space.

In one example of step 506, the non-fibrous bodies may be is in a compressed state (folded, rolled, bunched, etc.) to allow for insertion into the subject. In such embodiments, the non-fibrous bodies may be expanded or further compressed after insertion. Insertion can be facilitated by placing the subject in the Trendelenburg position, a vertical tilt, typically about 15°, with the feet higher than the head.

In one embodiment, step 506 may be executed to provide a barrier when the bowels are repositioned cephally, thereby providing a surgical space with access to the lower pelvic cavity. In another embodiment, step 506 may be executed to provide a barrier when the bowels are repositioned caudally, thereby providing a surgical space with access to the upper and middle abdominal cavity.

It is noted that in some embodiments, steps 504 and 506 may be combined, or step 506 may be executed before step 504. For example, the surgeon may position the non-fibrous absorbent bodies against the bowels and then reposition the bowels by manipulating the bowels through the non-fibrous absorbent bodies.

In certain embodiments of the present invention, the non-fibrous body or bodies are configured to be self-retaining. That is, at step 506, the body or collection of bodies are designed to provide a bowel retaining barrier without requiring additional instruments to maintain the surgical space. In other embodiments, the non-fibrous bodies are used in conjunction with retractor blades. In such embodiments, once the bodies are positioned within the subject abutting the bowels at step 506, the blades of a retractor are positioned against bodies to maintain the surgical space. In another embodiment, a net or other device is applied over the multiple non-fibrous bodies to hold the bodies in place.

In some embodiments of the invention, the non-fibrous bodies are utilized to absorb body fluids while also being used as a barrier between the bowels and the surgical space. Such body fluids may include, for example, blood of the subject. Further, in some embodiments, the non-fibrous absorbent body 100 is used to absorb body fluids even while not serving as a barrier between the bowels and the surgical space. Along these lines, FIG. 5B is another flow-chart of an exemplary method 550. At step 508, the bowels of a subject are accessed by a surgeon. Similar to the above embodiments, this may be done via a laparoscopic procedure or via a laparotomy. At step 510, a non-fibrous absorbent body is positioned to contact the bowels of the subject. At step 5120, the non-fibrous absorbent body is used to absorb body fluids proximate the bowels of the subject. In some embodiments, at least some of the method steps of FIG. 5B may be used in conjunction with a method of using the non-fibrous body as a barrier as detailed above with respect to FIG. 5A, and visa-versa.

In embodiments of the present invention, a non-fibrous body and method of use is provided where the body is sufficiently collapsible that body may be inserted into an abdomen via a laproscopic surgical procedure. In one such exemplary embodiment, the non-fibrous absorbent body may have general dimensions and configuration similar to, or the same as those detailed above, but also having sufficient collapsibility to be inserted into the abdominal cavity through a gel port or similar device or through a relatively small incision. Such an incision may be about one or about three to five inches in diameter. This as compared to the incision typically made through the ventral side of a subject in a more invasive procedure, which may result after retraction of the skin in an opening about 15 inches in diameter or more.

The non-fibrous body may be collapsed (e.g., rolled, folded or otherwise bunched together) to fit into the cannula of the trocar, small incision or gel port, etc. Sufficient force applied to the non-fibrous absorbent body causes the non-fibrous absorbent body to move through the cannula of the trocar and into the abdominal cavity. Once in the abdominal cavity, the non-fibrous body is uncollapsed (e.g., unrolled, unfolded, unbunchned, etc.) to expand to the configuration(s) detailed herein.

It is noted that in an exemplary embodiment, the non-fibrous body is collapsed in a manner to avoid kinking/creasing the material that causes a permanent deformation that interferes with the use of body. That is, an embodiment of the present invention entails collapsing the barrier and/or making the barrier from material that is substantially elastic. This may not be applicable to embodiments where the non-fibrous body is made entirely from a chamois, but may be applicable to embodiments where the non-fibrous body is reinforced by a support structure as detailed above.

Some exemplary embodiments of the non-fibrous body, as detailed herein, are collapsible from a relatively flattened state to a relatively cylindrical state without plastically deforming the non-fibrous body. This may be done by tightly rolling the non-fibrous body together. Also, embodiments of the non-fibrous body include a body that may be uncollapsed from the relatively cylindrical state to the relatively flattened state without plastically deforming the non-fibrous body. In some embodiments, this may be done remotely through a minimally invasive incision (e.g., that of a laparoscopic incision). Accordingly, some exemplary embodiments of the non-fibrous body as detailed herein are uncollapsible from a collapsed state (rolled state) to an uncollpased state (relatively flattened state) without plastically deforming the non-fibrous body. It is noted that in other embodiments, the collapsed state is a state different than or in addition to a rolled state (e.g., a rolled body that is also folded, a folded body, a bunched body, etc.)

As noted above, in an embodiment of the present invention, insertion of the non-fibrous bodies into the abdominal cavity may be accomplished by holding a portion of the collapsed non-fibrous body with a forceps and inserting the non-fibrous absorbent body through a minimally invasive incision (e.g., that of a laparoscopic incision)/gel port and into the abdominal cavity. Accordingly, an embodiment of the present invention includes the method steps of inserting an non-fibrous absorbent body in a collapsed state into the abdominal cavity through a laparoscopic incision and/or gel port, uncollapsing the non-fibrous body within the abdominal cavity by remotely manipulating the body through the incision/port made via the laparoscopy, and remotely positioning the non-fibrous body against the bowels.

Once the non-fibrous body is no longer needed in the abdomen, the barrier may be recollapse or collapsed to a new collapsed state (including separating portions of the non-fibrous body and removing those portions one or more at a time) so that the body may be withdrawn from the abdomen through the cannula of the trocar and/or through the incision in the abdomen.

The invention described and/or claimed herein is not to be limited in scope by the specific preferred embodiments herein disclosed, since these embodiments are intended as illustrations, and not limitations, of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims.

Reference herein to “one embodiment” or “an embodiment” means that a particular feature, structure, operation, or other characteristic described in connection with the embodiment may be included in at least one implementation of the invention. However, the appearance of the phrase “in one embodiment” or “in an embodiment” in various places in the specification does not necessarily refer to the same embodiment. It is further envisioned that a skilled person could use any or all of the above embodiments in any compatible combination or permutation.

What is claimed is:

1. A method of packing organs of a subject, comprising:
accessing an interior cavity of the subject;
repositioning the organs to provide a surgical space in the cavity; and
positioning a non-fibrous body against the bowels to provide a barrier between the organs and the surgical space.

2. The method of claim 1, wherein the non-fibrous body is absorbent.

3. The method of claim 1, wherein the non-fibrous body is a chamois.

4. The method of claim 3, wherein the chamois is a synthetic chamois.

5. The method of claim 3, wherein the chamois is a sterilized natural chamois.

6. The method of claim 1, wherein the non-fibrous body comprises a surface that is at least partially coated with one or more of an anti-viral and an anti-bacterial agent.

7. The method of claim 1, wherein positioning the non-fibrous body against the organs includes collapsing the non-fibrous body from a flattened state to a rolled state.

8. The method of claim 1, wherein positioning the non-fibrous body against the organs further includes:
positioning refractor blades abutting the non-fibrous body.

9. The method of claim 1, wherein a support frame is permanently attached to the non-fibrous body.

10. The method of claim 9, wherein the support frame is substantially elastically deformable.

11. The method of claim 9, wherein the frame comprises a wire mesh substantially surrounded by the non-fibrous body.

12. The method of claim 1, wherein the non-fibrous body has a generally rectangular shape when laid flat.

13. The method of claim 1, wherein the non-fibrous body is configured such that it has no abrasive edges.

14. The method of claim 1, wherein accessing the interior cavity comprises:
performing a laparoscopy.

15. The method of claim 14, further comprising:
inserting the non-fibrous body, in a collapsed state into the cavity through a gel port disposed in an incision of about four inch or less;
uncollapsing the non-fibrous body within the cavity by remotely manipulating the non-fibrous body through the gel port; and
positioning the non-fibrous body against the organs by remotely manipulating the non-fibrous body through the gel port.

16. The method of claim 1, wherein accessing the interior cavity comprises:
performing a laparotomy.

17. The method of claim 1, wherein the non-fibrous body substantially comprises a non-abrasive material.

18. The method of claim 1, further comprising:
positioning a plurality of non-fibrous bodies abutting the organs.

19. The method of claim 1, wherein the non-fibrous body is a foam material.

20. The method of claim 19, wherein the foam material is a silicone foam.

21. A surgical procedure, comprising:
accessing organs of a subject;
positioning a non-fibrous absorbent body in contact with the organs; and
absorbing body fluids proximate to the organs with the non-fibrous absorbent body.

22. The method of claim 21, wherein the non-fibrous absorbent body is a chamois.

23. The method of claim 21, wherein the chamois is a synthetic material.

24. The method of claim 21, wherein the chamois is a sterilized natural chamois.

25. The method of claim 21, wherein the non-fibrous absorbent body comprises a surface that is at least partially coated with at one or more of an anti-viral and an anti-bacterial agent.

26. The method of claim 21, wherein positioning the non-fibrous absorbent body in contact with the organs includes collapsing the non-fibrous absorbent body from a flattened state to a rolled state.

27. The method of claim 21, wherein positioning the non-fibrous absorbent body in contact with the organs further includes:
positioning retractor blades abutting the non-fibrous absorbent body.

28. The method of claim 21, wherein a support frame is permanently attached to the non-fibrous absorbent body.

29. The method of claim 27, wherein the support frame is substantially elastically deformable.

30. The method of claim 27, wherein the support frame comprises a wire mesh substantially surrounded by the absorbent body.

31. The method of claim 21, further comprising:
positioning a plurality of non-fibrous absorbent bodies in contact with the organs.

32. A device for packing organs of a subject, comprising:
a non-fibrous body; and
a support frame embedded in the body.

33. The device of claim 32, wherein the non-fibrous body is absorbent.

34. The device of claim 32, wherein the non-fibrous body is a chamois.

35. The device of claim 34, wherein the chamois is a silicone foam/synthetic chamois.

36. The device of claim 34, wherein the chamois is a sterilized natural chamois.

37. The device of claim 32, wherein a surface of the non-fibrous body is at least partially coated with one or more of an anti-viral and an anti-bacterial agent.

38. The device of claim 32, wherein the support frame is substantially elastically deformable.

39. The device of claim 32, wherein the support frame is a wire mesh.
